SEQUENCE LISTING (9 sequences)

SEQ ID NO: 1 - protein, 11 aa, Artificial Sequence
  P1  peptide
RTVVYPTMMFK

SEQ ID NO: 2 - protein, 11 aa, Artificial Sequence
  P1* peptide
RTVVYPTMMFK

SEQ ID NO: 3 - protein, 11 aa, Artificial Sequence
  P1 peptide
RTVVYPTMMFK

SEQ ID NO: 4 - protein, 11 aa, Artificial Sequence
  TAMRA-P1
RTVVYPTMMFK

SEQ ID NO: 5 - protein, 11 aa, Artificial Sequence
  TAMRA-P1
RTVVYPTMMFK

SEQ ID NO: 6 - protein, 10 aa, Artificial Sequence
  H3 peptide #10
PAKGTTSKRA

SEQ ID NO: 7 - protein, 10 aa, Artificial Sequence
  H3 peptide #11
PAKGTTSKRA

SEQ ID NO: 8 - protein, 9 aa, Artificial Sequence
  H3 peptide #12
AKGTTSKRA

SEQ ID NO: 9 - protein, 9 aa, Artificial Sequence
  H3 peptide #13
AKGTTSKRA

(19) United States
(12) Patent Application Publication
(10) Pub. No.: US 2010/0021939 A1
(43) Pub. Date: Jan. 28, 2010

(54) PROCESS FOR DETECTING ENZYME ACTIVITY IN AN IMMUNOASSAY

(76) Inventors: Joachim Krämer, Ellerbek (DE); Christiane Peiker, Halstenbek (DE); Karsten Henco, Erkrath (DE); Thomas Mander, Hamburg (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 12/505,680

(22) Filed: Jul. 20, 2009

Related U.S. Application Data
(62) Division of application No. 10/344,509, filed on Jul. 8, 2003, now abandoned.

(30) Foreign Application Priority Data
Aug. 11, 2000 (EP) 00117457.2
Aug. 24, 2000 (EP) 00118431.6
Oct. 18, 2000 (EP) 00122707.3
Dec. 21, 2000 (EP) 00128176.5

(51) Int. Cl. G01N 33/573; C07K 7/06

(52) U.S. Cl. 435/7.4; 530/328; 530/327

(57) ABSTRACT

The invention relates to a process for detecting enzyme activity in an immunoassay comprising the following steps:

a) providing a protein, a peptide, or a derivative thereof comprising the sequence motif

-Z-X-Y- or -Y-X-Z-

wherein
Z=an amino acid to be modified by the enzyme,
X=a sequence of amino acids, preferably between 0 and 1000 amino acids which may be the same or different,
Y=a discrimination enhancer for the binding to an antibody,
   as a substrate for the enzyme;
   b) incubating the protein, peptide, or derivative thereof with the enzyme to form a modified protein, peptide, or derivative thereof;
   c) adding an antibody discriminating the modified Z from the unmodified Z position of said protein, peptide, or derivative thereof, said discrimination being mediated by the presence of the affinity enhancer; and
   d) detecting the enzyme activity.

The present invention relates to a process for detecting enzyme activity in an immunoassay, in particular to a process for detecting dephosphorylation of phospho-serine or phospho-threonine (hereinafter also “phospho-serine/-threonine”)by the activity of a phosphatase as well as in particular to a process for detecting acetyltransferase or deacetylase activity in an immunoassay. The invention relates further to a kit for carrying out the assay and to a preferably luminescently labelled ligand.

In eukaryotic cells there are numerous post-translational modifications of proteins which are introduced after termination of the protein synthesis. In the post-translational modification of proteins the action of corresponding modification enzymes is required. There are a great number of post-translational modifications known which are introduced after termination of the protein synthesis.

As an example, the post-translational formation of disulfide bridges by the enzyme disulfide diisomerase may be mentioned. Similarly, particular chemical groups may be added to amino acids in a protein. Representative examples are the glycosylation, phosphorylation, acetylation, acylation and carboxylation. Also, particular chemical groups may be removed from a protein by processes like e.g. deglycosylation, dephosphorylation, deacetylation, deacylation and decarboxylation.

Reversible protein phosphorylation is an important mode of regulation of celLular processes. It is now apparent that protein phosphatases play in comparison to protein kinases an equally integral role in the control of cellular phospho-proteins. Concomitant control of kinases and phosphatases provides the cell with the capacity to rapidly switch proteins from their phosphorylated to dephosphorylated state to meet differing physiological demands.

Protein phosphatases are a diverse group of proteins that can be classified into two main families according to their substrate specificity: Serine/threonine phosphatases that dephosphorylate phospho-serine/-threonine residues and tyrosine phosphatases that dephosphorylate phospho-tyrosine residues. A third group, dual specificity protein phosphatases, which can dephosphorylate both phospho-serine/-threonine and phospho-tyrosine residues, actually belongs to the tyrosine phosphatase family.

Molecular cloning has identified many protein serine/threonine phosphatases. PP1 (or type 1 phosphatase) and PP2A make up more than 90% of the serine/threonine phosphatase activity in mammalian cells. PP1 phosphatases regulate a wide range of cellular processes, including cell-cycle progression, cell proliferation, protein synthesis, transcriptional regulation, and neurotransmission. PP1 is the major phosphatase that regulates glycogen metabolism in response to insulin and adrenalin. PP2A is supposed to function as a tumor suppressor.

To date, several dual specificity phosphatases have been identified in mammalian cells. As an example, CL100/3CH134 is shown to dephosphorylate threonine and tyrosine residues of extracellular signal-regulated kinases (ERK), thus leading to kinase inactivation. Since the initial cloning of CL100/3CH134, further dual specificity phosphatases have been identified. These include PAC1, hVH-2/MKP-2, hVH-3/B23 amongst others. These dual specificity phosphatases all appear to be effective in mediating inactivation of mitogen-activated protein (MAP) kinases (Camps, M. et. al, The FASEB Journal, 14, 6-16, 2000).

Detecting the activity of protein phosphatases would be useful for the high-throughput screening of chemical libraries. Modulators of phosphatase activity could eventually be developed into drugs used for the treatment of e.g. Parkinson's disease, Alzheimer's disease, cancer, or diabetis mellitus and other metabolic disorders. A review of known protein phosphatase inhibitors is given by Oliver, C. J. and Shenolikar, S. (Frontiers in Bioscience 3, d961-972, Sep. 1, 1998).

A further example of the importance of enzymatic modifications shall be given with view to acetyltransferases and deacetylases. The chromatin structure has an influence on key cellular processes such as DNA replication, transcription, DNA repair and differentiation. The chromatin structure and the binding of regulatory proteins to DNA can be modified by reversible acetylation of the tertiary amino groups of conserved lysine residues in the N-terminal tails of core histones. This enzymatic modification is controlled by histone acetyltransferases and histone deacetylases. Increasing evidence is accumulating that inhibitors of histone deacetylase may have a great potential in cancer therapy (Nakayama J. et al., Science, Vol. 292, 110-113, 2001; Darkin-Rattray et al., Proc. Natl. Acad. Sci. USA, Vol. 93, 13142-13147, 1996; Kölle et al., Methods: A companion to methods in enzymology, Vol. 15, 323-331, 1998). Inhibitors of histone deacetylase are of great potential as new drugs due to their ability to influence transcriptional regulation and to induce apoptosis or differentiation in cancer cells.

Due to the immense importance of enzymatic actions to pathogenic processes, up to now several assays for detecting enzymatic activities have been developed as shown by the following examples.

In a commercially available phosphatase assay, the release of phosphate from serine or threonine is detected by an absorbance change using Malachite Green as an indicator (Upstate Biotechnology, U.S.A., catalog numbers #14-110 and #14-111). This assay principle is a time consuming multi-step procedure and is highly sensitive to phosphate contaminations. Therefore, the different components of the phosphatase assay have to be carefully purified prior to the experiment.

The most frequently used phosphatase screens employ either radioactive substances or ELISAs. ELISAs are undesirable because they have a low throughput due to the extra steps required for both washing and the enzyme reaction. Radioactivity is used to detect dephosphorylation of phospho-serine or phospho-threonine by the activity of a phosphatase by the release of ³²P from the substrate peptide or protein (Current Protocols in Molecular Biology, Unit 18.2, John Wiley & Sons). However, there is a growing demand away from the use of radioactivity in assay applications, because of problems with costs, safety, disposal, and shelf-life of the ligand reagents.

Unlike dephosphorylation of tyrosine, the development of preferably homogeneous assays to detect the dephosphorylation phospho-serine/-threonine by phosphatases, has been impeded to date by the lack of anti-phospho-serine/phospho-threonine antibodies available that bind specifically, and with high affinity, to phospho-serine or phospho-threonine residues.

Also, so far no assay formats exist to measure the activity of histone deacetylases and potential modulators (i.e. inhibitors or activators) of said enzyme activity which are compatible with the requirements of high throughput screening, such as homogeneous (i.e. “mix, incubate and read”) assay format, high sensitivity and short measurement time.

From the foregoing, it will be clear that there is a continuing need for the development of cost-effective, facile and sensitive enzymatic assays for both high throughput screening (HTS) of potential drugs and secondary assays.

It was therefore a main object of the present invention to establish a generic assay method for detecting enzyme activity. In particular, it was an object to establish an assay method for detecting dephosphorylation of phospho-serine or phospho-threonine by phosphatases and to establish an assay method for detecting the activity of acetyltransferases and deacetylases. The assay methods should be highly reliable and simple to perform. In the case of phosphatases it should be usable without the need to develop specific high affinity anti-phospho-serine/-phospho-threonine antibodies.

The above mentioned main object has been solved by the assay process according to the features of claim 1.

The invention relates to a process for detecting enzyme activity in an immunoassay comprising the following steps:
a) providing a protein, a peptide, or a derivative thereof comprising the sequence motif

-Z-X-Y- or -Y-X-Z-

wherein
Z=an amino acid to be modified by the enzyme,
X 32 a sequence of amino acids, preferably between 0 and 1000 amino acids which may be the same or different,
Y=a discrimination enhancer for the binding to an antibody,
   as a substrate for the enzyme;
b) incubating the protein, peptide, or derivative thereof with the enzyme to form a modified protein, peptide, or derivative thereof;
c) adding an antibody discriminating the modified Z position from the unmodified Z position of said protein, peptide, or derivative thereof, said discrimination being mediated by the presence of the enhancer; and
d) detecting the enzyme activity.

Or to put it in other words, the invention relates to a method for determining the activity of an enzyme, comprising the steps of:
a) combining said enzyme with
   a protein, a peptide, or a derivative thereof comprising the sequence motif

-Z-X-Y- or -Y-X-Z-
wherein
Z=an amino acid to be modified by the enzyme,
X=a sequence of amino acids, preferably between 0 and 1000 amino acids, which may be the same or different,
Y=a discrimination enhancer for the binding to an antibody,
as a substrate for the enzyme; and
an antibody discriminating the modified Z position from the unmodified Z position of said protein, peptide, or derivative thereof, said discrimination being mediated by the presence of the enhancer;
b) detecting the enzyme activity.

By detecting the presence, absence or amount of a complex between the modified protein, peptide, or derivative thereof and the antibody, an enzyme activity can be measured.

In another aspect, the present invention relates to a process for detecting dephosphorylation of phospho-serine/-threonine by phosphatase activity in an immunoassay which comprises the following steps:
a) providing a protein, a peptide, or a derivative thereof comprising the sequence motif

-Z-X-Y- or -Y-X-Z-

wherein
Z=serine or threonine,
X=a sequence of amino acids, preferably between 0 and 1000 amino acids which may be the same or different,
Y=tyrosine, serine or threonine,
   as a substrate for the phosphatase, said protein, peptide, or derivative thereof being phosphorylated at the Z and Y position;
b) incubating the protein, peptide, or derivative thereof with the phosphatase to form a protein, peptide, or derivative thereof which is dephosphorylated at the Z position;
c) adding an antibody having a specificity to the protein, peptide, or derivative thereof that is phosphorylated in the Y and Z position; and
d) detecting the phosphatase activity.

In still another aspect, the present invention relates to a process for detecting acetylation or deacetylation of a substrate by virtue of acetylase or deacetylase enzyme activity, respectively, in an immunoassay which comprises the following steps:
a) providing a protein, a peptide, or a derivative thereof comprising the sequence motif

-Z-X-Y- or -Y-X-Z-

wherein
Z=an amino acid to be acetylated or deacetylated by the respective enzyme,
X=a sequence of amino acids, preferably between 0 and 1000 amino acids which may be the same or different,
Y=a discrimination enhancer for binding to an antibody,
   as a substrate for the enzyme;
b) incubating the protein, peptide, or derivative thereof with the enzyme to form a protein, peptide, or derivative thereof which is acetylated or deacetylated, respectively, at the Z position;
c) adding an antibody discriminating the modified Z position from the unmodified Z position of said protein, peptide, or derivative thereof, said discrimination being mediated by the presence of the enhancer; and
d) detecting the enzyme activity.

In the assay for detecting acetylase/deacetylase activity, the discrimination enhancer comprises preferably a phosphorylated amino acid.

Unless otherwise indicated, “phosphorylating activity” as used herein is synonymous with “kinase activity” and “dephosphory-lating activity” as used herein is synonymous with “phosphatase activity”. Similarly, unless otherwise indicated, a “kinase” is defined as a biological material capable of phosphorylating a peptide, protein, or derivative thereof, and a “phosphatase” is defined herein as a biological material capable of dephosphorylating a peptide, protein, or derivative thereof. The term “bis-phosphorylated” or “double phosphorylated” indicates that a protein, peptide or derivative thereof comprising the sequence motif -Z-X-Y- or -Y-X-Z- is phosphorylated both at the Z and Y position. Unless otherwise indicated, the term “bis-phosphorylated” or “double phosphorylated” as used herein does not exclude the possibility that said protein, peptide, or derivative thereof is also phosphorylated at positions other than Z and Y. Sometimes, the terms “discrimination enhancer” or “enhancer” will be used as synonymous to the term “affinity enhancer”.

The further subclaims define preferred embodiments of the process of the present invention.

In one embodiment, the substrate might be combined with said enzyme before the addition of said antibody. However, all assay components might also be combined simultaneously.

In the sequence motif, any amino acid or sequence of amino acids (X) can be inserted between Z and Y. The number of amino acids is preferably in the range of 0 to 1000 amino acids. A range of 0 to 1000 is preferred to include both linear and conformational antibody epitopes. X is particularly at least one amino acid, any other short amino acid sequences having at least two amino acids, such as oligopeptides, being also included. Preferably, X is proline, or glutamate, or glycine in the case of studying (de)phosphorylating enzyme activity on substrates related to JNK protein.

The antibody used is usually a monoclonal or polyclonal antibody. The antibody to be used discriminates the modified Z position from the unmodified Z position of the substrate.

According to the present invention, this discriminating capability is mediated by the presence of a discriminating enhancer in the substrate's amino acid sequence. The discriminating enhancer Y contained in the sequence motif has the function to promote and/or intensify the binding of an antibody which is added in step (c) of the process of the present invention. The enhancer Y can comprise at least one amino acid which might be modified or substituted, e.g. by a phosphate, sulphate, mono- or oligosaccharide, biotin, acetyl group, acyl group, hydroxyl group, thiol group, carboxyl group, carbonyl group or amide group. Usually the modifications or substitutions on the Z and Y groups are identical. It is, however, also possible that Z and Y comprise different modifications.

Depending on the enzyme modification reaction such as the enzymatic addition or elimination of groups to or from the protein, peptide, or derivative thereof, the enzyme may be of such kind that either the addition or the elimination of the chemical group is promoted by the enzyme. As examples, enzymes such as a kinase, phosphatase, sulphatase, carboxylase, decarboxylase, acylase, deacylase, hydroxylase, dehydroxylase, amidase, deamidase, acetylase or deacetylase may be mentioned.

In the event that the protein, peptide, or derivative thereof is enzyme-modified by addition of chemical groups to the Z position in the sequence motif, a corresponding co-substrate is used in step (b). Suitable co-substrates comprise, for example, a phosphate, sulphate, mono- or oligosaccharide, biotin, an acyl group, an acetyl group, a hydroxyl group, a thiol group, a carboxyl group, a carbonyl group or an amide group.

The immunoassay according to the present invention may be performed as a direct immunoassay, preferably a homogeneous direct binding immunoassay.

Any homogeneous assay is based on a mix and measure principle. This is highly advantageous over the conventional direct binding assays always requiring complicated separation steps of the assay components before detection.

In the direct binding immunoassay, typically either a detectable peptide/protein/derivative thereof, or a detectable TV antibody is used. Usually, detection takes place by optical methods and any beforehand labelling of the peptide etc. or the antibody may be carried out according to conventional standard techniques. Preferably, the peptide/protein/derivative thereof and/or the antibody are labelled using a luminescent or a radioactive tag or by using specific labelling molecules such as a reporter enzyme or an affinity ligand.

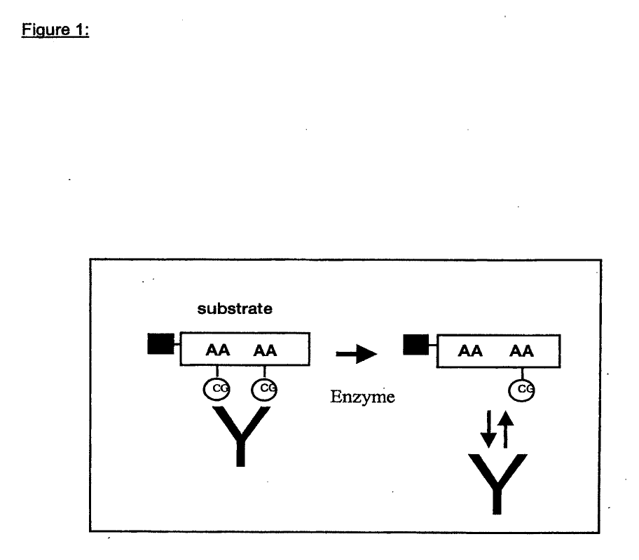

In FIG. 1 a schematic drawing of one embodiment of the direct assay of the present invention is shown. A labelled modified protein/peptide substrate carries two chemical groups (CG) on two amino acids (AA) in the Z and Y position. By the action of an enzyme, one of the CGs is cleaved off. Consequently, the labelled modified substrate does no longer bind to the specific antibody.

The assay according to the present invention may also be performed as a competition immunoassay, preferably a homogeneous indirect binding assay. Also, a homogeneous indirect binding assay is based on the mix and measure principle.

In this indirect binding assay, a detectable ligand is added to compete with either the modified or the unmodified form of the peptide/protein/derivative for binding to an antibody. The ligand is preferably made optically detectable or labelled using a luminescent or radioactive tag or by using specific labelling molecules such as a reporter enzyme or an affinity ligand.

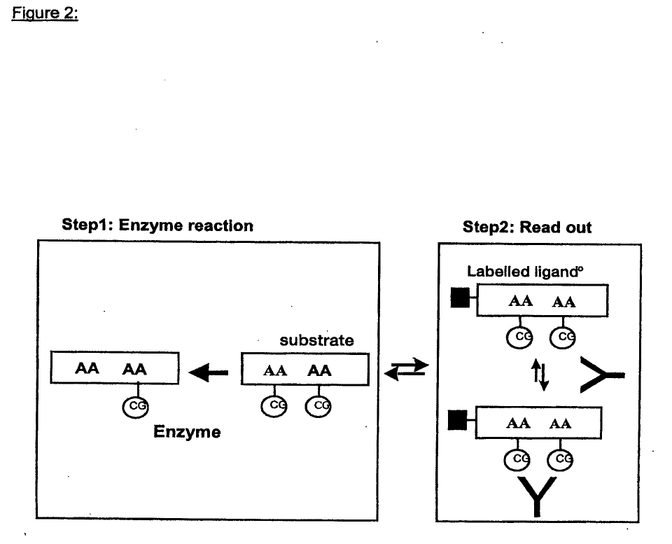

FIG. 2 shows a schematic drawing of an embodiment of the indirect assay of the invention. In a first step, a modified peptide/protein/substrate having two chemical groups (CG) on two amino acids (AA) in the Z and Y position is subjected to the action of an enzyme, consequently loosing one of the chemical groups. The product of the reaction does no longer compete with the ligand for binding to the specific antibody.

The assay of the present invention can be performed as a fluorescence immunoassay. In particular, fluorescence immunoassays such as a fluorescence polarization (FP) immunoassay, a fluorescence correlation spectroscopy (FCS) assay, a fluorescence lifetime (FL) assay, or a fluorescence intensity distribution analysis (FIDA) assay may be mentioned.

The present invention also concerns a kit for detecting enzyme activity in an immunoassay according to the present invention. Said kit comprises the following components:
(i) a substrate comprising the sequence motif

-Z-X-Y- or Y-X-Z-

wherein
Z=an amino acid to be modified by the enzyme,
X=a sequence of amino acids, preferably between 0 and 1000 amino acids which may be the same or different,
Y=a discrimination enhancer for the binding to an antibody; and
(ii) an antibody discriminating the modified Z from the unmodified Z position of said substrate, said discrimination being mediated by the presence of the discrimination enhancer.

In a preferred embodiment, said kit further comprises an enzyme, reaction buffers, and/or a co-substrate.

In still a further embodiment, the kit comprises a detectable ligand, preferably a luminescent ligand, said ligand comprising the sequence motif

-Z-X-Y- or -Y-X-Z-

wherein
Z=an amino acid to be modified by the enzyme,
X=a sequence of amino acids, preferably between 0 and 1000 amino acids which may be the same or different,
Y=a discrimination enhancer for the binding to antibody.

Such a kit is particularly suited for performing competitive assays according to the present invention. The present invention also relates to the said ligand as such, i.e. without being offered in combination with the other kit components.

As will be outlined in more detail below, the invention also relates to an assay for detecting phosphatase activity. Therefore, in another aspect a kit is provided which is suited for performing such assay. The kit for detecting phosphatase activity in an immunoassay comprises the following components:
(i) a substrate comprising the sequence motif

-Z-X-Y- or Y-X-Z-

wherein
Z=serine or threonine,
X=a sequence of amino acids, preferably between 0 and 1000 amino acids which may be the same or different,
Y=tyrosine, serine or threonine,
said substrate being phosphorylated at the Z and Y position; and
(ii) an antibody having a specificity to said substrate.

This kit might also comprise an enzyme (in this case a phosphatase such as serine or threonine phosphatase, or a dual specificity tyrosine phosphatase) and reaction buffers. For the application of the kit in the performance of competitive assays, the kit might include a detectable, e.g. luminescent ligand (as competitor) comprising the sequence motif

-Z-X-Y- or Y-X-Z-

wherein
Z=serine or threonine,
X=a sequence of amino acids, preferably between 0 and 1000 amino acids which may be the same or different,
Y=tyrosine, serine or threonine,
   said ligand being phosphorylated at the Z and Y position. The present invention also relates to the said ligand as such, i.e. without being offered in combination with the other kit components.

As will also be outlined in more detail below, the invention also relates to a process for detecting acetylase or deacetylase activity. Therefore, also a specific kit is provided for performing the corresponding assays. Such a kit comprises the following components:
(i) a substrate comprising the sequence motif

-Z-X-Y- or Y-X-Z-

wherein
Z=an amino acid to be acetylated or deacetylated by a respective enzyme,
X=a sequence of amino acids, preferably between 0 and 1000 amino acids which may be the same or different,
Y=a discrimination enhancer for the binding to an antibody, said discrimination enhancer preferably comprising a phosphorylated amino acid; and
(ii) an antibody discriminating the modified Z from the unmodified Z position of said substrate, said discrimination being mediated by the presence of the discrimination enhancer.

The processes as well as the kits according to the present invention may be used for screening modulators (inhibitors or activators) for enzyme activity. Such modulators may play a key role in metabolism of animals including human beings. They are considered to be extremely useful for the treatment of diseases, such as metabolic disorders and cancer.

As an example, the assay of the present invention can be carried out to detect phosphatase activity. By detecting the presence, absence, or amount of a complex between a bis-phosphorylated protein/peptide/derivative thereof and an antibody, phosphatase activity can be measured.

In the following a particular example of a process for detecting dephosphorylation of phospho-serine or phospho-threonine by the activity of a phosphatase will be shown. In this case:
in the protein/peptide/derivative thereof Z is chosen to be serine or threonine whereas Y is chosen to be tyrosine, serine or threonine, said protein/peptide being phosphorylated at the Y and Z position;
a phosphatase is used to form a protein/peptide/derivative thereof which is dephosphorylated at the Z position;
the antibody has a specificity to the protein/peptide/derivative thereof that is phosphorylated in the Y and Z position;
the activity detected is phosphatase activity.

In this exemplary assay, X in the sequence motif typically comprises proline, or glutamate, or glycine.

Any protein containing the above motif may be used. For example, a protein such as the bis-phosphorylated JNK protein is preferably used, which is the c-Jun N-terminal kinase, and which is also known in the literature as the stress-activated protein kinase 1 (SAPK1). For example, bis-phosphorylated (sometimes also referred to as “activated”) JNK1, JNK2 or JNK3 protein may be mentioned. An antibody directed against bis-phosphorylated JNK protein is sometimes referred to as “anti-active JNK antibody” or “JNK antibody”.

When it is more convenient to use a peptide substrate, the peptide sequence is preferably selected from the active-site loop, e.g. for PP1 or PP2A from the JNK1/2/3 active site. For instance, said peptide for PP1 or PP2A comprises or is composed of the amino acid sequence H-Lys-Phe-Met-Met-pThr-Pro-pTyr-Val-Val-Thr-Arg-NH₂, wherein p means phosphorylated.

When the incubation of the protein or peptide is carried out in the presence of a serine or threonine phosphatase, the phosphatase is preferably a serine/threonine protein phosphatase type 2A (PP2A) or type 2B (PP2B). In a further preferred embodiment, the incubation might be carried out using PP1, PP4 (also named PPX) or PP6 (also named PPV) as a phosphatase.

However, it is also possible to perform the assay utilising a tyrosine phosphatase with double specificity. Examples for such phosphatases comprise CL100/3CH134, PAC1, hVH-2/MKP-2, hVH-3/B23, hVH-5, MKP-3/PYST1, B59, MKP-4, and MKP-5.

For studying e.g. the (de)phosphorylation of JNK substrates, it has been shown that a particularly preferred antibody, is a polyclonal antibody specific for bis-phosphorylated JNK substrates. Such antibodies are commercially available. It has been revealed that the antibody specifically recognizes a phosphorylated threonine or serine residue at the Z position within both a synthetic substrate peptide or JNK, said recognition being mediated by the presence of the discrimination enhancer.

The process of the present example as well as the corresponding kit and the detectable ligand may be used for screening for specific modulators of serine or threonine or dual specificity phosphatase activity. The assay is particularly suitable for screening compound libraries in order to locate molecules which inhibite or activate phosphatases. These molecules may be promising candidates for designing drugs used for the treatment of e.g. metabolic disorders and cancer.

The accompanying figures illustrate the present invention. Abbreviations for the described peptides are used as follows (p means phosphorylated):
P1° H-Lys-Phe-Met-Met-Thr-Pro-pTyr-Val-Val-Thr-Arg-NH2
P1* H-Lys-Phe-Met-Met-pThr-Pro-Tyr-Val-Val-Thr-Arg-NH2
P1*° H-Lys-Phe-Met-Met-pThr-Pro-pTyr-Val-Val-Thr-Arg-NH2 (sometimes also referred to as P1°*)
TAMRA-P1*° 5-TAMRA-AEEA-Lys-Phe-Met-Met-pThr-Pro-pTyr-Val-Val-Thr-Arg-NH2 (sometimes also referred to as TAMRA-P1°*)
TAMRA 5′-(6-carboxytetramethylrhodamine)
AEEA 8-amino-3,6-dioxaoctanoic acid linker

In the figures the following is shown:

FIG. 1 is a schematic drawing of the principle of the direct assay of the present invention.

FIG. 2 is a schematic drawing of the principle of the indirect assay of the present invention.

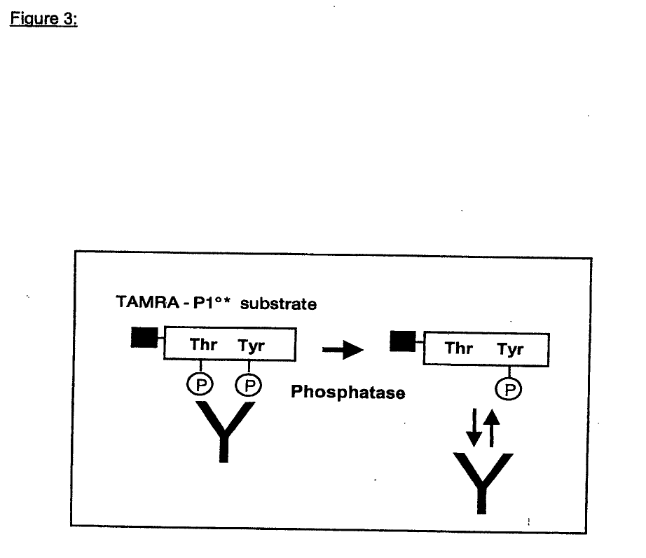

FIG. 3 is a schematic drawing of a preferred embodiment of the principle of the direct assay of the present invention to detect phosphatase activity. The-TAMRA-P1°* substrate becomes dephosphorylated at the threonine residue in the presence of the phosphatase. Upon dephosphorylation, the TAMRA-labelled P1°*-peptide will no longer bind to the polyclonal anti-active JNK antibody.

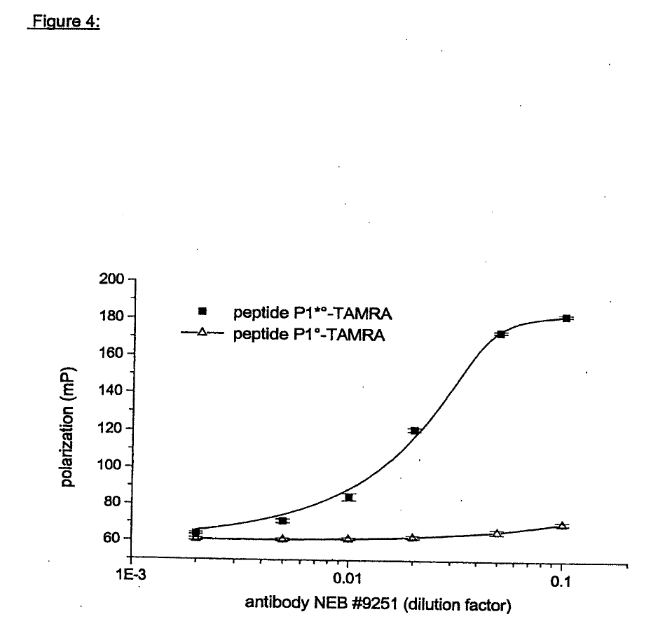

FIG. 4 is a diagram showing the specific binding of the polyclonal JNK antibody to TAMRA-labelled P1°*-peptide; as a control, no binding is measured for TAMRA-labelled P1°-peptide.

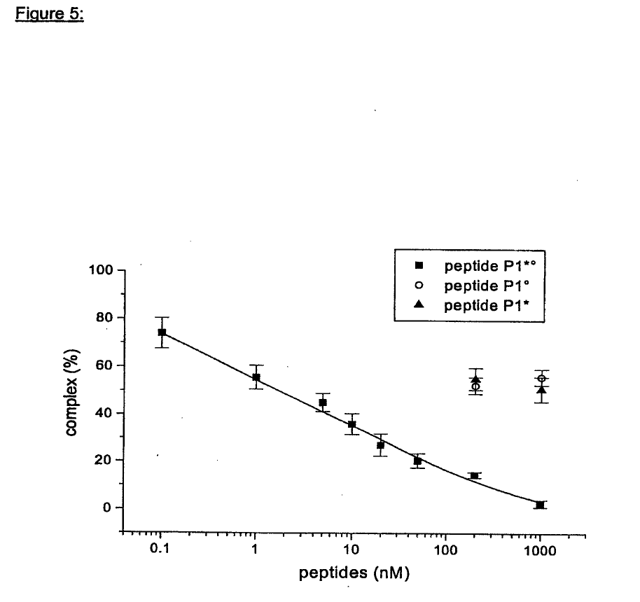

FIG. 5 is a diagram showing the competition of the binding of the polyclonal JNK antibody to the TAMRA-labelled P1°*-peptide using double-phosphorylated P1°*-peptide (determination of the IC50 for the P1°*-peptide competitor) and, as controls, mono-phosphorylated P1°- and P1*-peptide.

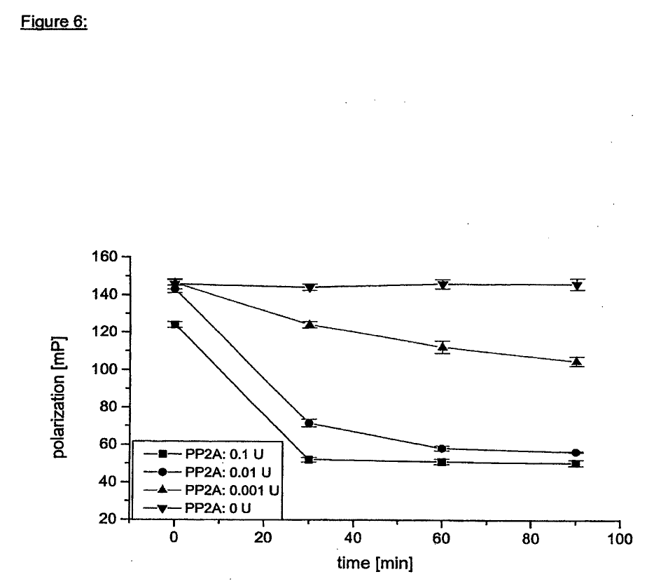

FIG. 6 is a diagram of the results of a direct phosphatase assay (see FIG. 3) showing a time course of the dephosphorylation of the TAMRA-P1°*-peptide at different PP2A phosphatase concentrations. Desphosphorylated TAMRA-P1°*-peptide does not bind to the polyclonal JNK antibody, resulting in low fluorescence polarization.

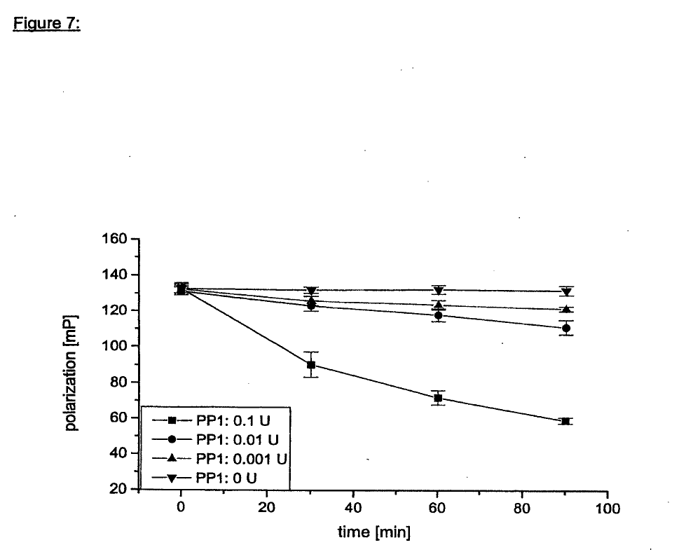

FIG. 7 is a diagram of the results of a direct phosphatase assay (see FIG. 3) showing a time course of the dephosphorylation of the TAMRA-P1°*-peptide at different PP1 phosphatase concentrations. Desphosphorylated TAMRA-P1°*-peptide does not bind to the polyclonal JNK antibody, resulting in low fluorescence polarization.

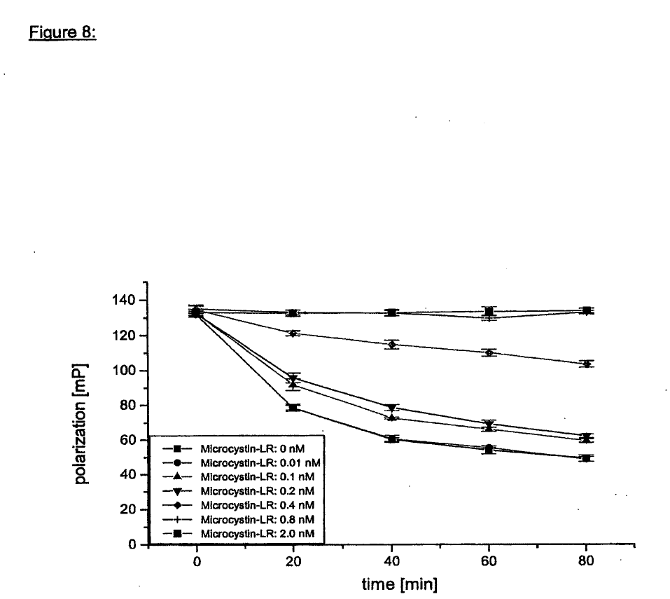

FIG. 8 is a diagram of the results of a direct phosphatase assay (see FIG. 3) showing the inhibition of the dephosphorylation of the TAMRA-P1°*-peptide by PP2A phosphatase at different microcystin-LR (phosphatase inhibitor) concentrations. Desphosphorylated TAMRA-P1°*-peptide does not bind to the polyclonal JNK antibody, resulting in low fluorescence polarization.

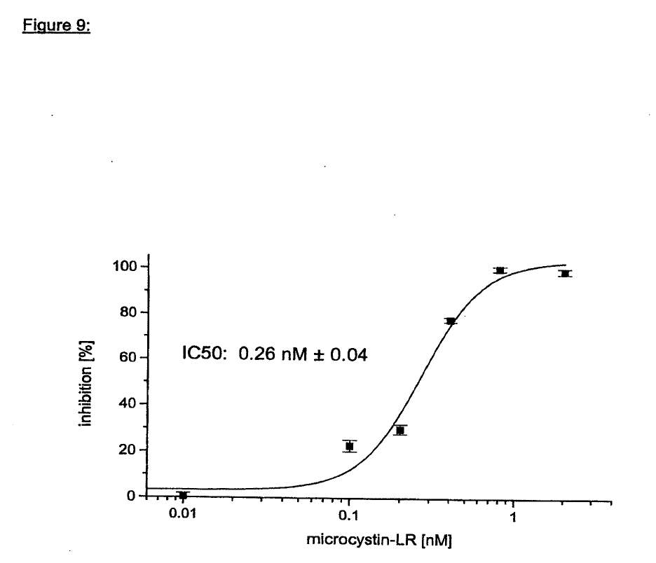

FIG. 9 is a diagram showing the inhibition of the PP2A-dependent dephosphorylation of the TAMRA-P1°*-peptide substrate by the phosphatase inhibitor microcystin-LR. From the inhibition curve, the half-maximal inhibition concentration (IC50) is calculated.

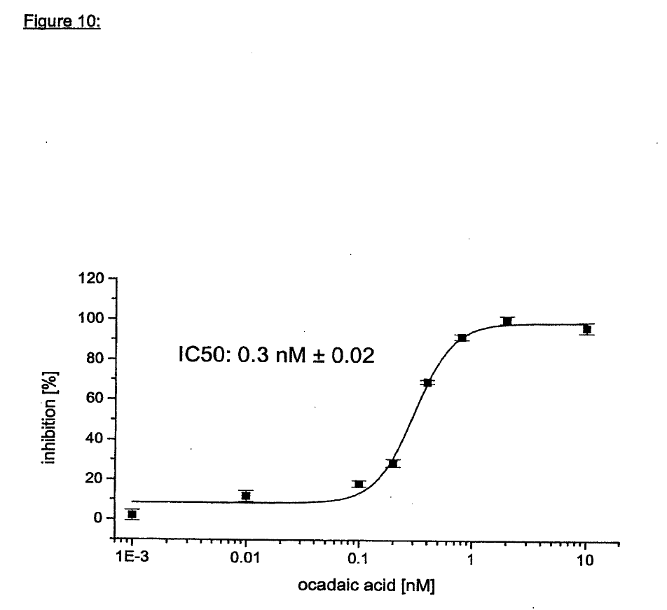

FIG. 10 is a diagram showing the inhibition of the PP2A-dependent dephosphorylation of the TAMRA-P1°*-peptide substrate by the phosphatase inhibitor ocadaic acid. From the inhibition curve, the half-maximal inhibitory concentration (IC50) is calculated.

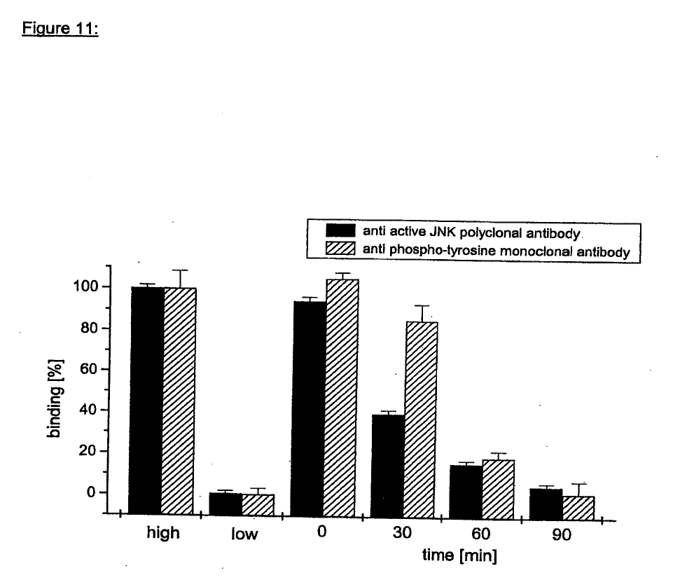

FIG. 11 is a diagram of the results of a direct phosphatase assay (see FIG. 3) showing a time course of the dephosphorylation of the TAMRA-P1°*-peptide by PP1 phosphatase which is detected by two different antibodies. The polyclonal anti-active JNK antibody detects both the desphosphorylation of the phospho-threonine and/or the dephosphorylation of the phospho-tyrosine. As a control, the monoclonal anti-phospho-tyrosine antibody only detects the dephosphorylation of the phospho-tyrosine. Both events result in low fluorescence polarization.

The controls denoted as “high” mean maximum binding of the antibodies to the TAMRA-P1°* peptide. The controls denoted as “low” mean no binding of the antibodies to the TAMRA-P1°* peptide due to addition of 200 nM P1°* competitor peptide.

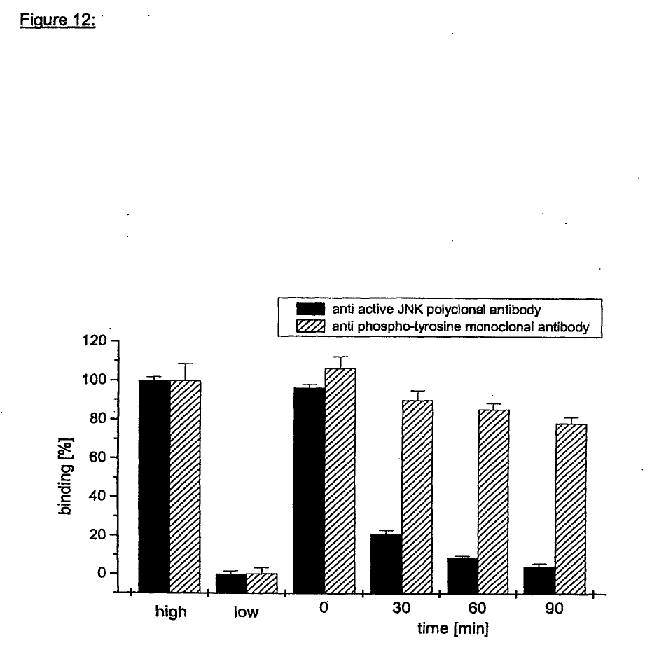

FIG. 12 is a diagram of the results of a direct phosphatase assay (see FIG. 3) showing a time course of the dephosphorylation of the TAMRA-P1°*-peptide by PP2A phosphatase which is detected by two different antibodies. The polyclonal anti-active JNK antibody detects both the desphosphorylation of the phospho-threonine and/or the dephosphorylation of the phospho-tyrosine. The monoclonal anti-phospho tyrosine antibody only detects the dephosphorylation of the phospho-tyrosine. Both events result in low fluorescence polarization. The controls denoted as “high” mean maximum binding of the antibodies to the TAMRA-P1°* peptide. The controls denoted as “low” mean no binding of the antibodies to the TAMRA-P1°* peptide due to addition of 200 nM P1°* competitor peptide.

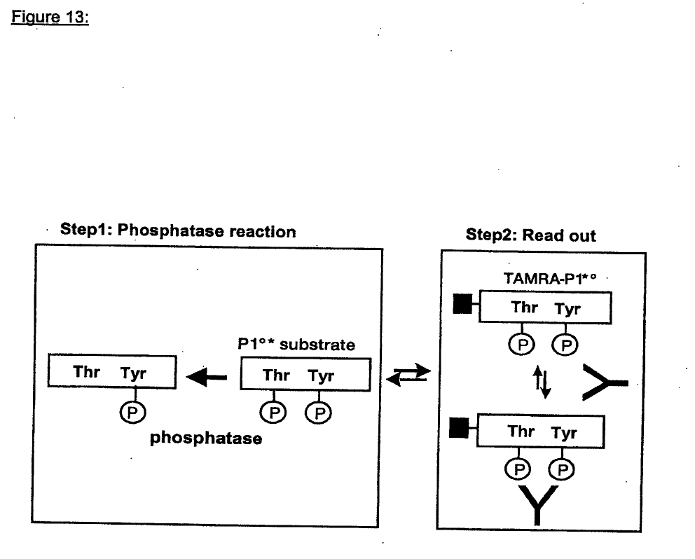

FIG. 13 is a schematic drawing of the principle of the indirect phosphatase assay of the present invention. In a first step, the bis-phosphorylated non-fluorescent P1°*-peptide is dephosphorylated by the activity of a phosphatase. The product of the reaction (dephosphorylated P1°* peptide) will no more compete with the TAMRA-labelled P1°*-peptide for the binding to the polyclonal anti-active JNK antibody which are both added in a second step (stop solution including a reagent inactivating the phosphatase).

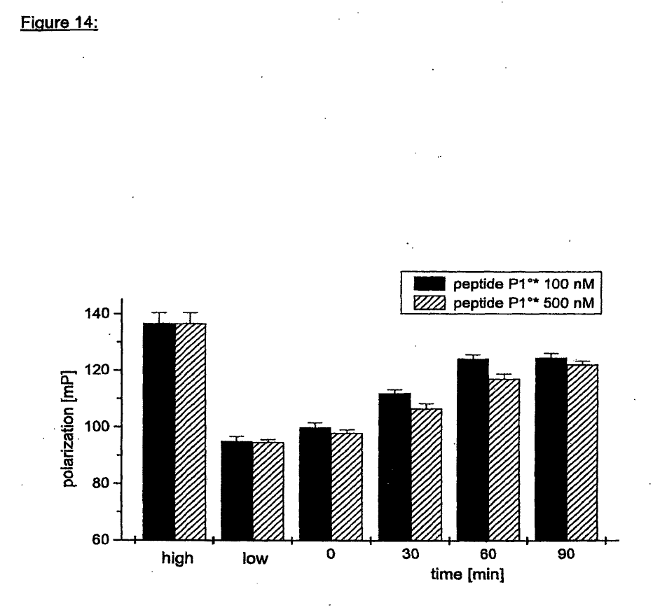

FIG. 14 is a diagram of the results of an indirect phosphatase assay (see FIG. 13) showing a time course of the dephosphorylation of the P1°*-peptide substrate by PP2A phosphatase at different P1°*-peptide concentrations. Desphosphorylated P1°*-peptide does no more compete with the TAMRA-labelled P1°*-peptide for the binding to the polyclonal JNK antibody, resulting in high fluorescence polarization. The controls denoted as “high” mean maximum binding of the antibodies to the TAMRA-P1°* peptide. The controls denoted as “low” mean no binding of the antibodies to the TAMRA-P1°* peptide due to the presence of 100 nM or 500 nM P1°* substrate peptide.

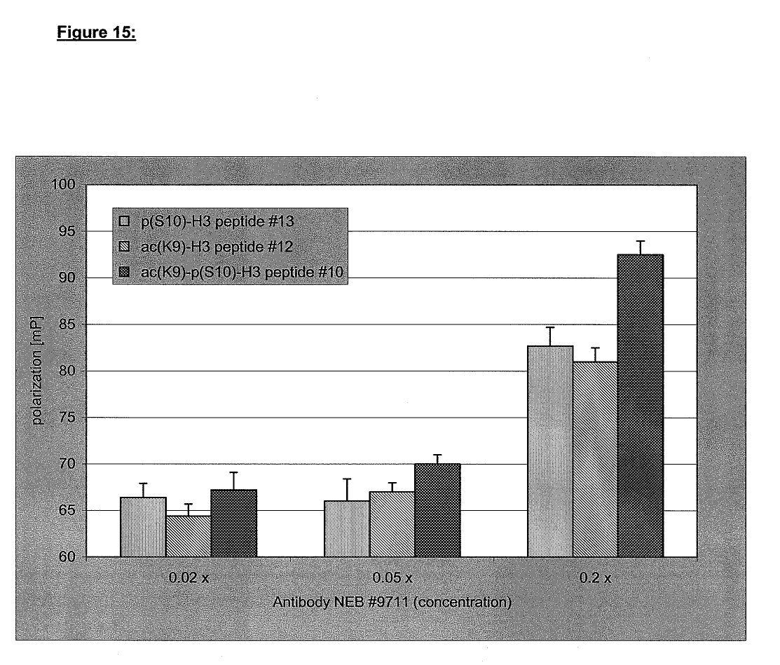

FIG. 15 is a schematic drawing showing the binding of an ac(K9)-p(S10)-specific antibody from New England Biolabs. (#9711) to dual-modified ac(K9)p(s10)-H3 Peptide #10; as a control, only low binding is measured for mono-modified ac(K9)-H3 peptide #12 and p(S10)-H3 Peptide #13.

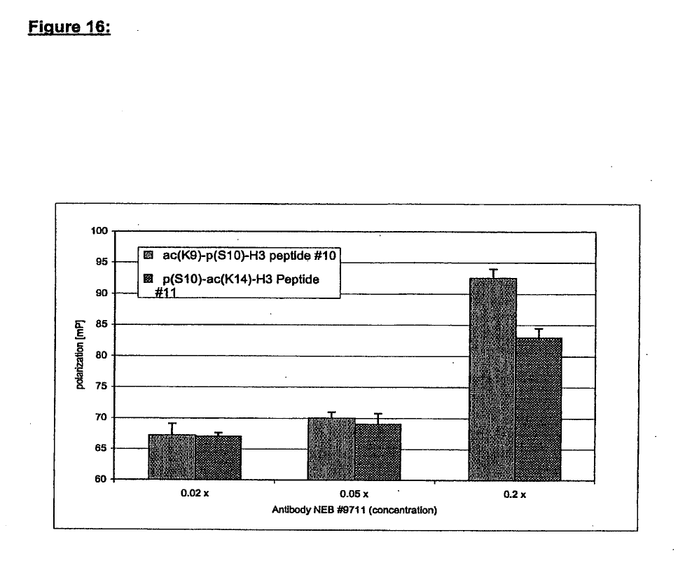

FIG. 16 is a schematic drawing showing the binding of an Li ac(K9)-p(S10)-specific antibody from New England Biolabs (#9711) to dual-modified ac(K9)p(S10)-H3 Peptide #10; as a control, only low binding is measured for dual-modified p(S10)ac(K14)-H3 Peptide #11.

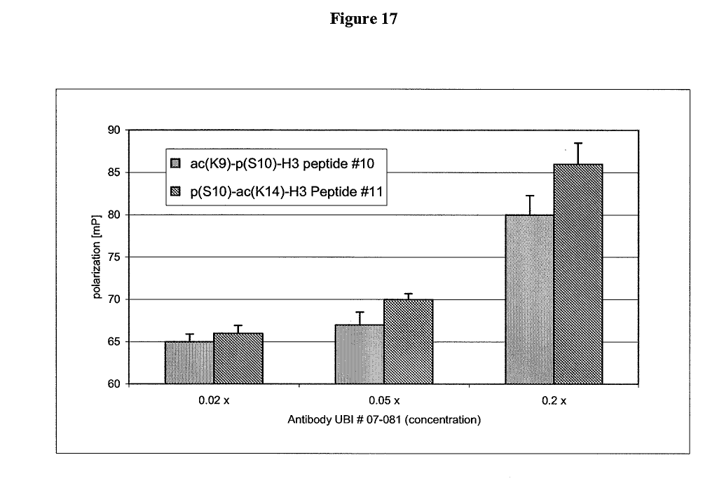

FIG. 17 is a schematic drawing showing the binding of an p(S10)-ac(K14)-specific antibody from Upstate Biotech (#07-081) to dual-modified p(S10)ac(K14)-H3 peptide #11; as a control, only low binding is measured for dual-modified ac(K9)p(S10)-H3 peptide #10.

The following experiments further illustrate the present invention.

### EXAMPLE 1

### Measurement of the Binding Affinity (KD) of anti-active JNK Antibody (New England Biolabs NEB #9251)

This polyclonal antibody detects all three isoforms of the SAPK1/JNK proteins only when they are activated by dual phosphorylation at threonine₁₈₃/tyrosine₁₈₅. Binding of polyclonal anti active JNK antibody #9251 (concentration: 0.02 mg/ml, equiv. 130 nM) to TAMRA-labelled P1*° peptide (at 5 nM) was measured at different antibody concentrations by fluorescence polarization. As a control, no binding of the antibody to the monophosphorylated TAMRA-P1° peptide (at 5 nM) is detected.

Affinity of polyclonal anti-active-JNK antibody for peptide P1*°-TAMRA: K_{D}=2.6 nM

The results of this experiment are illustrated in FIG. 4. It has been revealed that the commercially available polyclonal anti-active JNK-specific antibodies are particularly useful in the assay of the present invention. As can be seen in FIG. 4, the polyclonal anti-active-JNK antibody detects the peptides—derived from the active site loop of JNK protein—only when dual phosphorylated at threonine₁₈₃ and tyrosine₁₈₅. The polarization values dramatically increase when using the double-phosphorylated TAMRA-P1*° peptide in contrast to mono-phosphorylated labelled TAMRA-P1° peptide (sequences see below).

### EXAMPLE 2

### Determination of ICSO for Peptide P1°*

Competition of NEB #9251 antibody-P1°*-TAMRA complex with P1°* peptide was measured by fluorescence correlation spectroscopy: Binding of antibody NEB #9251 (1:20 diluted) to TAMRA-labelled peptide P1*° (5 nM) was competed with non-fluorescent, double-phosphorylated peptide P1*°. Control peptides: mono-phosphorylated P1° and P1* did note compete effectively.

Peptide P1*°: IC₅₀=5.0 nM+3.3 nM

The results of this experiment are illustrated in FIG. 5. In FIG. 5 the binding of the same polyclonal antibody to TAMRA-labelled peptide P1*° (5 nM) in competition with the peptides P1° (mono-phosphorylated, sequence see below), peptide P1* (mono-phosphorylated, sequence see below) or peptide P1*° (double phosphorylated, sequence see below) is presented. As can be deduced from the complex formation, only bis-phosphorylated peptide P1*° is able to compete effectively with the TAMRA-labelled peptide P1*° (peptide ligand) for binding to the antibody while the other mono-phosphorylated peptides displace less than 50% of the bound ligand even at high concentrations (up to 1 μM).

### EXAMPLE 3

### Dephosphorylation of TAMRA-P1°* Peptide by PP2A Phosphatase

In FIG. 6 the rate of dephosphorylation of the P1°*-TAMRA substrate peptide by PP2A phosphatase is determined at various PP2A concentrations. Efficient dephosphorylation of the P1°*-TAMRA substrate peptide using the direct phosphatase assay is performed as follows: In a total volume of 80 μl the P1°*-TAMRA substrate peptide (at 10 nM) is dephosphorylated using 0.1, 0.01, or 0.001 units of PP2A. The phosphatase reactions are incubated at 30° C. and aliquots (20 μl) are withdrawn after 0 min, 30 min, 60 min and 90 min and mixed with 10 μl of a stop solution containing the following reagents (all final concentrations): hydrogenperoxide 10 mM, polyclonal anti active JNK antibody from NEB at a dilution of 1:20.

The formed dephosphorylated P1°*-TAMRA peptide product can be detected as it does not bind to the polyclonal phospho-JNK antibody. As a result, a drop of the fluorescence polarization is detected which is inversely proportional to PP2A phosphatase activity.

### EXAMPLE 4

### Dephosphorylation of TAMRA-P1°* Peptide by PP1 Phosphatase

In FIG. 7 the rate of dephosphorylation of the P1°*-TAMRA substrate peptide by PP1 phosphatase is detected at various PP1 concentrations using the same direct assay as described in example 3. Efficient dephosphorylation of the P1°*-TAMRA substrate peptide using the direct phosphatase assay is performed as follows: In a total volume of 80 μl the P1°*-TAMRA substrate peptide (at 10 nM) is dephosphorylated using 0.1, 0.01, or 0.001 units of PP1. The phosphatase reactions are incubated at 30° C. and aliquots (20 μl) are withdrawn after 0 min, 30 min, 60 min and 90 min and mixed with 10 μl of a stop solution containing the following reagents (all final concentrations): hydrogenperoxide 10 mM, polyclonal anti active JNK antibody from NEB (1:20 dilution). The formed dephosphorylated P1°*-TAMRA peptide product can be detected as it does not bind to the polyclonal anti active JNK antibody. As a result, a drop of the fluorescence polarization is detected which is inversely proportional to PP1 phosphatase activity.

### EXAMPLE 5

### Inhibition of the PP2A Phosphatase-Dependent Dephosphorylation of TAMRA-P1°* Peptide by Microcystin-LR

In FIG. 8 the inhibition of the dephosphorylation of P1°*-TAMRA substrate peptide by PP2A phosphatase is determined for the phosphatase inhibitor microcystin-LR. The inhibition of PP2A phosphatase is detected using the same direct assay as described in example 3. As a result, no reduction of the binding of the TAMRA-labelled P1*° peptide to the polyclonal anti active JNK antibody is detected at high microcystin-LR concentrations due to full inhibition of PP2A.

Assay conditions: PP2A phosphatase (0.004 units) is incubated with P1°*-TAMRA peptide (10 nM) and microcystin-LR (0, 0.01, 0.1, 0.2, 0.4, 0.8, 2 nM) in a total volume of 100 μl at 30° C. At different time points aliquots are withdrawn from the enzyme reaction and mixed with hydrogen peroxide (10 nM), polyclonal anti active JNK antibody from NEB #9251 (1:20 dilution).

### EXAMPLE 6

### Inhibition of the PP2A Phosphatase-Dependent Dephosphorylation of TAMRA-P1°* Peptide by Microcystin-LR

FIG. 9 shows the inhibition of the dephosphorylation of P1°*-TAMRA substrate peptide by PP2A phosphatase using the phosphatase inhibitor microcystin-LR. The half-maximal inhibitory concentration (IC50) for microcystin-LR is calculated from the data of FIG. 8.

### EXAMPLE 7

### Inhibition of the PP2A Phosphatase-Dependent Dephospharylation of TAMRA-P1°* Peptide by Ocadaic Acid

FIG. 10 shows the inhibition of the dephosphorylation of P1°*-TAMRA substrate peptide by PP2A phosphatase using the phosphatase inhibitor ocadaic acid. The half-maximal inhibitory concentration (IC50) for ocadaic is calculated. IC50=0.3 nM +/− 0.02 nM.

Assay conditions: PP2A phosphatase (0.003 units) is incubated with P1°*-TAMRA peptide (10 nM) and ocadaic acid (0, 0.001, 0.01, 0.1, 0.2, 0.4, 0.8, 2, 10 nM) at 30° C. At different time points aliquots are withdrawn from -the enzyme reaction and mixed with hydrogen -peroxide (10 nM), polyclonal anti active JNK antibody from NEB #9251 (1:20 dilution).

### EXAMPLE 8

### Measurement of the Specificity of PP1 Phosphatase Using Two Different Antibodies

FIG. 11 shows the detection of PP1 phosphatase-dependent dephosphorylation of TAMRA-P1°* Peptide using the polyclonal anti active JNK antibody (NEB #9251) and the monoclonal anti phospho-tyrosine antibody (NEB #9411). The dephosphorylation of the TAMRA-P1°* peptide by PP1 phosphatase is performed using the same direct assay as described in FIG. 7, with the only exception that two antibodies with different specificities were used for the read-out. As a result, a PP1 phosphatase-dependent dephosphorylation of the phospho-threonine and the phospho-tyrosine is detected. PP1 has a lower specificity when compared to PP2A (see example 9).

Assay conditions: PP1 phosphatase (0.1 units) is incubated with P1°*-TAMRA peptide (10 nM) at 30° C. At different time HI points aliquots are withdrawn from the enzyme reaction and mixed with the stop solution containing hydrogen peroxide (10 nM) along with either the polyclonal anti active JNK antibody from NEB #9251 (1:20 dilution) or the monoclonal anti phospho-tyrosine antibody from NEB #9411 (1:100 dilution)

### EXAMPLE 9

### Measurement of the Specificity of PP2A Phosphatase Using Two Different Antibodies

FIG. 12 shows the detection of PP2A phosphatase-dependent dephosphorylation of TAMRA-P1°* Peptide using the polyclonal anti active JNK antibody NEB #9251 and the monoclonal anti phospho-tyrosine antibody NEB #9411. The dephosphorylation of the TAMRA-P1°* peptide by PP2A phosphatase is performed using the same direct assay as described in FIG. 7, with the only exception that two antibodies with different specificities were used for the read-out. As a result, a PP2A phosphatase-dependent dephosphorylation of the phospho-threonine is detected. PP2A has a high specificity for phospho-threonine.

Assay conditions: PP2A phosphatase (0.006 units) is incubated with P1°*-TAMRA peptide (10 nM) at 30° C. At different time points aliquots are withdrawn from the enzyme reaction and mixed with the stop solution containing hydrogen peroxide (10 nM) along with either the polyclonal anti active JNK antibody from NEB #9251 (1:20 dilution) or the monoclonal anti phospho-tyrosine antibody from NEB #9411 (1:100 dilution)

### EXAMPLE 10

### Dephosphorylation of Non-Fluorescent P1°* Peptide by PP2A Phosphatase (Indirect Phosphatase Assay, see FIG. 13)

FIG. 14 shows the detection of PP2A phosphatase-dependent dephosphorylation of the P1°* substrate peptide in an indirect phosphatase assay.

In a first step (enzyme reaction), the bisphosphorylated P1°* substrate peptide is incubated with PP2A. In a second step (stop solution, including hydrogenperoxide), the polyclonal anti active JNK antibody (NEB #9251) is added together with TAMRA-P1°* peptide. As a result of the PP2A activity, the P1°* substrate peptide is dephosphorylated and does no longer compete with the TAMRA-P1°* peptide for the binding to the polyclonal anti active JNK antibody. This can be detected by an increase in fluorescence polarization.

Assay conditions: PP2A phosphatase (0.5 units) is incubated with 100 nM or 500 nM P1°* substrate peptide in a total volume of 80 μl at 30° C. At different time points, aliquots (20 μl) are withdrawn from the enzyme reaction and mixed with 10 μl of the stop solution containing hydrogen peroxide (10 nM), TAMRA-P1°* peptide at (5 nM) together with the polyclonal anti active JNK antibody from NEB (1:20 dilution).

### List of Reagents of the Described Phosphatase Assays;

### List of Used Peptides:

TAMRA-P1*° 5-TAMRA-AEEA-Lys-Phe-Met-Met-pThr-Pro-pTyr-Val-Val-Thr-Arg-NH₂

T MRA-P1° 5-TAMRA-AEEA-Lys-Phe-Met-Met-Thr-Pro-pTyr-Val-Val-Thr-Arg-NH₂

P1*° H-Lys-Phe-Met-Met-pThr-Pro-pTyr-Val-Val-Thr-Arg-NH₂

P1° H-Lys-Phe-Met-Met-Thr-Pro-pTyr-Val-Val-Thr-Arg-NH₂

P1* H-Lys-Phe-Met-Met-pThr-Pro-Tyr-Val-Val-Thr-Arg-NH₂

### Bisphosphorylated Substrate Peptide:

TAMRA-P1*°-Peptide 5-TAMRA-AEEA-Lys-Phe-Met-Met-pThr-Pro-pTyr-Val-Val-Thr-Arg-NH₂

Supplier: EVOTEC BioSystems AG

solid-phase peptide synthesis

HK-03-65-P1-13; M=2089 g/mol; MALDI (2092.18)

1 mM stock solution in 100% DMSO

### Bisphosphorylated Competitor Peptide:

P1*°-Peptide: H-Lys-Phe-Met-Met-pThr-Pro-pTyr-Val-Val-Thr-Arg-NH₂

Supplier: EVOTEC BioSystems AG

solid-phase peptide synthesis

HK-03-60-P1-7; M=1532 g/mol; MALDI (1531.88)

10 mM stock solution in 100% DMSO

### Monophosphorylated Control Peptides

P1°-Peptide: H-Lys-Phe-Met-Met-Thr-Pro-pTyr-Val-Val-Thr-Arg-NH₂

Supplier: EVOTEC BioSystems AG

solid-phase peptide synthesis

HK-03-58-HF; M=1451.g/mol; MALDI (1453.57)

10 mM stock solution in 100% DMSO

P1*-Peptide: H-Lys-Phe-Met-Met-pThr-Pro-Tyr-Val-Val-Thr-Arg-NH₂

Supplier: EVOTEC BioSystems AG

solid-phase peptide synthesis

HK-03-33-HF; M=1452 g/mol; MALDI (1452.23)

10 mM stock solution in 100% DMSO

### Enzymes:

PP1 phosphatase:

Supplier: Upstate Biotech cat. no. 14-110, lot no. 18524

PP2A phosphatase:

Supplier: Upstate Biotech cat. no. 14-111, lot no. 20574

### Anti Active JNK-Specific Antibody:

New England Biolabs, U.S.A.: NEB. #9251

Anti Phospho-Tyrosine-Specific Monoclonal Antibody pY100:

New England Biolabs, U.S.A.: NEB #9411

### Reagents and Buffers

### Assay Buffer:

10× HEPES-Assay-Buffer pH 7.2

500 mM HEPES

100 mM MgCl₂

10 mM DTT

Dissolve 77 mg DTT (FLUKA cat. no. 43815; MW 154.25 g/mol) [final concentration 10 mM] in 30 ml of Millipore water, add 5 ml of 1M MgCl₂ [final concentration 100 mM], and 5.96 g HEPES (FLUKA cat. no. 54457) [final concentration 500 mM] and adjust pH with 1M NaOH to pH 7.2. Top up to 50 ml with Millipore water. The buffer will be filtered sterile (0.22 μm), aliquoted to 1.5 ml and should be stored at −20° C.

### 1M MgCl₂:

Dissolve 10.2 g MgCl₂.6H₂O (FLUKA cat. no. 63068) in 50 ml of Millipore water. Buffer should be filtered (0.42 μm) and can be stored at 4° C. for several months.

1% (w/v) Puatonic F-127

Dissolve 0.5 g Pluronic (Sigma, cat. no. P-2443) in 50 ml of Millipore water. Stir gently to get a clear solution. Solution should be filtered (0.42 μm) and can be stored at 4° C. for several months.

### 1× BEPES-Assay-Buffer/0.1% Pluronic/0.01 BSA pH 7.5: (2 ml):

50 mM HEPES

10 mM MgCl₂

1 mM DTT

0.1% Pluronic

0.01% BSA

Add 50 μl 4% (w/v) Pluronic F-127 in water, add 200 μl of 10× HEPES-Assay-Buffer pH 7.5 and add 20 μl of 1% BSA in water (Merck Albumin Fraktion V) to 2 ml of Millipore water.

1% (w/v) Albumin Fraktion V (BSA)

Dissolve 0.1 g Albumin Fraktion V (Merck cat.no.1.12018.0100) in 10 ml of steril filtered (0.22 μm) Millipore water and aliquote to 100 μl. The stock should be stored at −20° C.

DMSO: 100% DMSO

The solvent was purchased from SIGMA (cat. No. P-2650), sterile filtered.

### Hydrogenperoxide 30% (H₂O₂)

Add 10 μl of 30% H₂O₂ (Merck cat.no.8.22287.1000) in 78 μl 1× HEPES-Assay-Buffer/0.1% Pluronic/0.01% BSA pH 7.5 (final concentration 1M).

### Microcystin-LR:

Supplier: Sigma cat.no. M-2912, lot-no. 48H1100

### Ocadaic Acid:

Supplier: Sigma cat.no. 0-7760, lot no. 129H1199

### EXAMPLE 11

### Acetylation of Lysines in Proteins

The histone deacetylase assay uses fluorescently-labelled peptides (TAMRA dye as a label) derived from histone H3 sequence. In the assay two different polyclonal antibodies are used which bind specifically to acetylated lysine redidues (ac(K) at positions ac(K9) or ac(K10)). Acetylation of a single lysine results in a relative small change of the molecular structure of the aminoacid side chain. The resulting change in the binding affinity to acetyl-specific antibodies is in general difficult to detect in a homogeneous binding assay. According to the invention, therefore an additional modification (phosphorylation of serine at position (S10)) was included within the H3-peptide sequence to enhance the affinity of the peptides to the applied antibodies. From the results of three independing binding experiments (FIGS. 15-17) it is evident that dual-modified peptide conjugates confer a higher affinity for the applied antibodies when compared to mono-modified peptide conjugates. This characteristic is of great value to measure the different acetylation states of lysine residues using acetyl-specific antibodies. In principle, the described binding assay can be applied both for the detection of acetyltransferase or deacetylase activity, in particular histone acetyltransferase/deacetylase.

### Reagents and Buffers:

### Fluorescent TAMRA-Peptide Conjugates:

All fluorescent peptides were supplied by Evotec BioSystems AG. Amino acids are given in the one-letter code. Ac denotes acetylated, p denotes phosphorylated.

ac(K9)p(S10)-H3 peptide #10:

TAMRA-A-R-ac(K9)-p(S10)-T-T-G-K-A-P

p(S10)ac(K14)-H3 peptide #11:

TAMRA-A-R-K-p(S10)-T-T-G-ac(K14)-A-P

ac(K9)-H3 peptide #12:

TAMRA-A-R-ac(K9)-S-T-T-G-K-A

p(S10)-H3 peptide #13:

TAMRA-A-R-K-p(S10)-T-T-G-K-A

### Antibodies:

1. Polyclonal ac(K9)-p(S10)-specific antibody from New England Biolabs, U.S.A., (#9711)

2. Polyclonal p(S10)-ac(K14)-specific antibody from Upstate Biotech, U.S.A., (#07-081)

### Assay Buffer:

10× HEPES-Assay-Buffer pH 7.5

500 mM HEPES

1 mM EGTA

100 mM DTT

62.5 mM NaCl

Dissolve 770 mg DTT (FLUKA cat. no. 43815; MW 154.25 g/mol) [final concentration 100 mM] in 30 ml of Millipore water, add 625 μl of 5M NaCl₂ [final concentration 62.5 mM], 19.02 mg EGTA (Merck cat. No.1.06404; MW 380.35 g/mol) [final concentration 1 mM] and 5.96 g HEPES (FLUKA cat. no. 54457) [final concentration 500 mM] and adjust pH with 1M NaOH to pH 7.5. Top up to 50 ml with Millipore water. The buffer will be filtered sterile (0.22 μm) aliquoted to 1.5 ml and should be stored at −20° C.

### 1M MgCl₂:

Dissolve 10.2 g MgCl₂×6H₂O (FLUKA cat. no. 63068) in 50 ml of Millipore water. Buffer should be filtered (0.42 μm) and can be stored at 4° C. for several months.

1% (w/v) Pluronic F-127

Dissolve 0.5 g Pluronic (Sigma, cat. no. P-2443) in 50 ml of Millipore water. Stir gently to get a clear solution. Solution should be filtered (0.42 μm) and can be stored at 4° C. for several months.

1× HEPES-Assay-Buffer/0.1% Pluronic pH 7.5: (15 ml):

50 mM HEPES

0.1 nM EGTA

10 mM DTT

0.1% Pluronic

Add 1.5 ml 1% (w/v) Piuronic F-127 in water and 1.5 ml of 10× HEPES-Assay-Buffer pH 7.5. to 12 ml of Millipore water. Stir the mixture and check the pH value. The mixture could be stored at 4° C. for 1-2 weeks.

### 0.5M EDTA (50 ml):

Dissolve 9.309 g EDTA (Roche Molecular Biochemicals, cat. no. 808288) in 40 ml of Millipore water and adjust pH to 8-9 with 10M NaOH in order to get a clear solution. Top up to 50 ml. with Millipore water. Buffer should be filtered (0.42 μm) and can be stored at 4° C. for several months.

### 10 mM ATP (15 ml):

Dissolve 9.1 mg ATP (Roche Molecular Biochemicals, cat. no. 126888) in 1.5 ml 1× HEPES-Assay-Buffer/0.1% Pluronic pH 7.5. The solution should be filtered (0.42 μm) and can be stored at −20° C. for 2 weeks.

### DMSO: 100% DMSO

The solvent was purchased from SIGMA (cat. No. P-2650), sterile filtered.

1-41. (canceled)

42. A process for detecting acetylase or deacetylase enzyme activity, respectively, in an immunoassay comprising the following steps:
a) providing a protein, peptide or derivative thereof comprising the sequence motif
-Z-X-Y- or -Y-X-Z-
wherein
Z=an amino acid to be acetylated or deacetylated by the respective enzyme,
X=a sequence of amino acids, preferably between 0 and 1000 amino acids which may be the same or different,
Y=a discrimination enhancer for the binding to an antibody,
as a substrate for the enzyme;
b) incubating the protein, peptide, or derivative thereof with the enzyme to form a protein, peptide or derivative thereof which is acetylated or deacetylated, respectively, at the Z position;
c) adding an antibody capable of discriminating the modified Z position from the unmodified Z position of said protein, peptide, or derivative thereof, said discrimination being mediated by the presence of the enhancer; and
d) detecting the enzyme activity.

43. The process according to claim 42 wherein the discrimination enhancer comprises a phosphorylated amino acid.

44. A kit for detecting acetylase or deacetylase activity in an immunoassay, comprising the following components:
a substrate as defined in claim 42; and
an antibody as defined in claim 42.

45. The kit according to claim 44 further comprising a detectable ligand, preferably a luminescent ligand, said ligand comprising the following sequence motif
-Z-X-Y- or -Y-X-Z-
wherein
Z=serine or threonine
X=a sequence of amino acids, preferably between 1 and 1000 amino acids which may be the same or different,
Y=tyrosine, serine or threonine,
said ligand being phosphorylated at the Y and Z positions.

46. A detectable ligand for detecting an enzyme activity in an immunoassay comprising the sequence motif according to claim 44.

47. The kit according to claim 44 further comprising an enzyme, reaction buffers, and/or a co-substrate.

48. A process for detecting acetylase or deacetylase enzyme activity, respectively, in an immunoassay, comprising the following steps:
i) providing substrate for an enzyme selected from the group consisting of a protein, a peptide and a derivative thereof comprising the sequence motif
-Z-X-Y- or -Y-X-Z
wherein
Z=an amino acid to be acetylated or deacetylated by the respective enzyme,
X=a sequence of amino acids, preferably between 0 and 1000 amino acids which may be the same or different,
Y=a discrimination enhancer for the binding to an antibody;
(ii) incubating the protein, peptide, or derivative thereof with the enzyme to form a protein, peptide or derivative thereof which is modified by a modification selected from the group consisting of acetylation and deacetylation at the Z position;
(iii) adding an antibody capable of discriminating the modified Z position from the unmodified Z position of said protein, peptide, or derivative thereof, said discrimination being mediated by the presence of the enhancer; and
(iv) detecting the enzyme activity in a homogeneous assay.

49. The process according to claim 48 wherein the process steps are performed using a timing selected from the group consisting of sequentially and at least partly simultaneously.

50. The process according to claim 48 wherein the discrimination enhancer comprises a phosphorylated amino acid.

51. A process for screening for modulators of acetylase or deacetylase enzyme activity, respectively, in an immunoassay, comprising the following steps:
i) providing substrate for an enzyme selected from the group consisting of a protein, a peptide and a derivative thereof comprising the sequence motif
-Z-X-Y- or -Y-X-Z
wherein
Z=an amino acid to be acetylated or deacetylated by the respective enzyme,
X=a sequence of amino acids, preferably between 0 and 1000 amino acids which may be the same or different,
Y=a discrimination enhancer for the binding to an antibody;
(ii) providing a potential modulator of acetylase or deacetylase enzyme activity;
(ii) incubating the protein, peptide, or derivative thereof with the enzyme and potential modulator to form a protein, peptide or derivative thereof which is modified by a modification selected from the group consisting of acetylation and deacetylation at the Z position;
(iii) adding an antibody capable of discriminating the modified Z position from the unmodified Z position of said protein, peptide, or derivative thereof, said discrimination being mediated by the presence of the enhancer; and
(iv) detecting the enzyme activity in a homogeneous assay.

52. A kit for detecting acetylase or deacetylase activity in an immunoassay, comprising the following components:
a substrate as defined in claim 51;
an antibody as defined in claim 51;
at least one potential modulator of an acetylase or deacetylase enzyme.

53. The kit according to claim 52 further comprising a detectable ligand, preferably a luminescent ligand, said ligand comprising the following sequence motif
-Z-X-Y- or -Y-X-Z-
wherein
Z=serine or threonine,
X=a sequence of amino acids, preferably between 1 and 1000 amino acids which may be the same or different,
Y=tyrosine, serine or threonine,
said ligand being phosphorylated at the Y and Z positions.

54. A detectable ligand for detecting an enzyme activity in an immunoassay comprising the sequence motif according to claim 52.

55. The kit according to claim 52 further comprising an enzyme, reaction buffers, and/or a co-substrate.
